(19) United States
(12) Patent Application Publication
(10) Pub. No.: US 2013/0165779 A1
(43) Pub. Date: Jun. 27, 2013

(54) TEMPERATURE ESTIMATION METHOD AND TEMPERATURE ESTIMATION APPARATUS USING THE SAME

(71) Applicant: Samsung Electronics Co., Ltd., Suwon-si (KR)

(72) Inventors: Ji-young PARK, Yongin-si (KR); Ki-wan CHOI, Yongin-si (KR); Dong-geon KONG, Yongin-si (KR); Hyoung-ki LEE, Yongin-si (KR)

(73) Assignee: Samsung Electronics Co., Ltd., Suwon-si (KR)

(21) Appl. No.: 13/676,478

(22) Filed: Nov. 14, 2012

(30) Foreign Application Priority Data
Dec. 27, 2011 (KR) 10-2011-0143926

(51) Int. Cl. A61B 8/00; A61B 5/01

(52) U.S. Cl. CPC A61B 8/00; A61B 5/015
USPC 600/438; 600/549

(57) ABSTRACT

An accuracy-improved temperature estimation method includes measuring a physical attribute of a tissue based on a reference signal and a received signal, a first estimation operation to estimate a first temperature of the tissue based on the physical attribute, calculating at least one parameter used in a bio heat transfer model based on the estimated first temperature, and a second estimation operation to estimate a second temperature of the tissue by using the calculated at least one parameter and the bio heat transfer model.

### CROSS-REFERENCE TO RELATED APPLICATIONS

This application claims the priority benefit of Korean Patent Application No. 10-2011-0143926, filed on Dec. 27, 2011, in the Korean Intellectual Property Office, the disclosure of which is incorporated herein in its entirety by reference.

### BACKGROUND

1. Field

The following description relates to a temperature estimation method, and a temperature estimation apparatus using the same, and more particularly, to a method of estimating a temperature of a tissue used during a non-invasive therapy, and a temperature estimation apparatus using the method.

2. Description of the Related Art

Along with the development of medical science, non-invasive surgeries have been recently performed, rather than minimally invasive surgeries, in order to apply a topical treatment to a tumor. A High Intensity Focused Ultrasound (HIFU) method, an example of a non-invasive surgery, is a method of removing a tumor by heating the tumor with high ultrasound energy focused on the tumor to cause necrosis of the tumor. In this case, for safe and efficient necrosis of the tumor, it is important to monitor a temperature change of a tissue according to the applied energy.

### SUMMARY

A first temperature estimation method may provide accurate results when a tumor is initially heated, but as the actual temperature of the tumor increases above a predetermined value, the accuracy of the results obtained from the first estimation method may decrease. Accordingly, to receive more accurate results, a second temperature estimation method may be used when the temperature increases above the predetermined value. The second temperature estimation method may use the results obtained from the first temperature estimation method as a parameter in additional calculations.

The following description relates to an accuracy-improved method of estimating a temperature of a tissue, and a temperature estimation apparatus using the method.

Additional aspects will be set forth in part in the description which follows and, in part, will be apparent from the description, or may be learned by practice of the presented embodiments.

According to an aspect of the following description, a temperature estimation method includes measuring a physical attribute of a tissue based on a reference signal and a received signal, a first estimation operation to estimate a first temperature of the tissue based on the physical attribute, calculating at least one parameter used in a bio heat transfer model based on the estimated first temperature, and a second estimation operation to estimate a second temperature of the tissue by using the calculated at least one parameter and the bio heat transfer model.

The first estimation operation may be performed when a temperature of the tissue exists in a first range, and the second estimation operation may be performed when the temperature of the tissue exists in a second range greater than the first range.

The second estimation operation may be performed when the first temperature estimated in the first estimation operation is greater than a predetermined value.

The temperature estimation method may further include a non-invasive therapy operation to remove a lesion by heating the tissue, wherein at least one of the first estimation operation and the second estimation operation is performed at the same time as the non-invasive therapy operation.

The at least one parameter may include at least one of heat conductivity of the tissue and heat applied to the tissue from the outside of the tissue.

In the parameter calculation, the at least one parameter may be calculated by substituting the estimated first temperature that is less than a predetermined value and a temperature variation into the bio heat transfer model.

The parameter calculation may include generating a spatio-temporal temperature map of the tissue based on the first temperature of the tissue, and calculating the at least one parameter based on intermediate expressions obtained by substituting the spatio-temporal temperature map into the bio heat transfer model.

The at least one parameter may be calculated by applying a least square method to the intermediate expressions.

The physical attribute measurement may be performed using at least one of a Harmonic Motion Imaging (HMI) method, a Change in Backscattered Energy (CBE) method, an echo shift method, and a nonlinear B/A parameter method as a method of estimating a temperature by measuring a speed variation, a magnitude variation, and a form change due to a nonlinear characteristic with respect to an ultrasound signal, or performed using a method of estimating a temperature by directly using a sensor.

The physical attribute measurement may include radiating an ultrasonic wave to a predetermined area corresponding to the tissue of a person to be diagnosed, receiving an echo ultrasonic wave reflected by the tissue, and converting the echo ultrasonic wave to the received signal.

The physical attribute measurement may be performed using an echo shift method, and the physical attribute may be determined by a speed variation of the reference signal and the received signal.

The second estimation operation may include generating a temperature model of the tissue by substituting the calculated at least one parameter into the bio heat transfer model, and estimating the second temperature of the tissue based on the temperature model.

According to an aspect of the following description, a temperature estimation method includes radiating an ultrasonic wave to a predetermined area corresponding to a tissue of a person to be diagnosed, receiving an echo ultrasonic wave reflected by the tissue, converting the echo ultrasonic wave to a received signal, measuring an echo shift between a reference signal and the received signal, a first estimation operation to estimate a first temperature of the tissue based on the echo shift, calculating at least one parameter used in a bio heat transfer model based on the estimated first temperature; and a second estimation operation of estimating a second temperature of the tissue by using the at least one parameter and the bio heat transfer model, the second estimation operation being performed when the estimated first temperature is greater than a predetermined value.

The temperature estimation method may further include outputting a temperature of the tissue based on at least one of the first temperature and the second temperature.

In the temperature output, the first temperature may be output as the temperature of the tissue when the estimated first temperature is less than the predetermined value, and the second temperature may be output as the temperature of the tissue when the estimated first temperature is equal to or greater than the predetermined value.

According to an aspect of the following description, a temperature estimation apparatus includes a measurement unit to measure a physical attribute of a tissue based on a reference signal and a received signal, a first estimation unit to estimate a first temperature of the tissue based on the physical attribute, and a second estimation unit to estimate a second temperature of the tissue by using a bio heat transfer model modeled based on the estimated first temperature.

A temperature estimation method may include measuring a physical attribute of a issue, estimating a first temperature of the tissue based on the measured physical attribute, determining if the first temperature is below a predetermined value, and selectively outputting the first temperature if the first temperature is below the predetermined value, or if the first temperature is not below the predetermined value, outputting a second temperature calculated from a bio heat transfer model.

The bio heat transfer model may use the first temperature as a parameter.

### BRIEF DESCRIPTION OF THE DRAWINGS

These and/or other aspects will become apparent and more readily appreciated from the following description of the embodiments, taken in conjunction with the accompanying drawings in which:

FIG. 1 is a flowchart illustrating a temperature estimation method according to an embodiment;

FIG. 2 is a graph showing temperatures of a tissue, which are estimated using the temperature estimation method of FIG. 1;

FIG. 3 is a flowchart illustrating a temperature estimation method according to an embodiment;

FIG. 4 is a schematic diagram of an ultrasonic treatment device and a temperature estimation apparatus according to embodiments;

FIG. 5, part (a) is a graph showing an echo ultrasonic wave received before a temperature of a tissue increases;

FIG. 5, part (b) is a graph showing an echo ultrasonic wave received after the temperature of the tissue increases;

FIG. 5, part (c) is a graph showing a result obtained by measuring an echo shift between the echo ultrasonic waves shown in FIG. 5, parts (a) and (b);

FIG. 5, part (d) is a graph showing a variation rate of the echo shift of FIG. 5, part (c);

FIG. 6 is a graph showing a relationship between temperatures of tissues and velocities of propagation of echo ultrasonic waves;

FIG. 7 is a graph showing a relationship between an echo shift and a first temperature of the tissue estimated in a first estimation operation;

FIGS. 8 and 9 are flowcharts illustrating a calculation operation of the temperature estimation method according to embodiments;

FIGS. 10 and 11 illustrate parameters calculated in the calculation operation of FIG. 8;

FIGS. 12 and 13 illustrate results obtained by estimating a temperature of the tissue by using the temperature estimation method according to an embodiment;

FIG. 14 illustrates a first temperature estimated by using an echo shift method when a temperature of the tissue is high;

FIG. 15 illustrates graphs showing the temperature estimation result of FIG. 13 in more detail; and

FIG. 16 is a block diagram of the temperature estimation apparatus according to an embodiment.

### DETAILED DESCRIPTION

The present inventive concept will now be described more fully with reference to the accompanying drawings, in which exemplary embodiments of the invention are shown.

The embodiments are provided to fully describe the present inventive concept to those of ordinary skill in the art, may be embodied in many different forms, and should not be construed as being limited to the embodiments set forth herein; rather, these embodiments are provided so that this disclosure will be thorough and complete, and will fully convey the concept of the present inventive concept to those of ordinary skill in the art.

The terminology used in the application is used only to describe specific embodiments and does not have any intention to limit the inventive concept. An expression in the singular includes an expression in the plural unless they are clearly different from each other in a context. In the application, it should be understood that terms, such as “comprise” and/or “comprising”, are used to indicate the existence of implemented feature, number, step, operation, element, part, and/or a combination of them without excluding in advance the possibility of existence or addition of one or more other features, numbers, steps, operations, elements, parts, and/or combinations of them. As used herein, the term “and/or” includes any and all combinations of one or more of the associated listed items.

Although terms, such as ‘first’ and ‘second’, can be used to describe various elements, areas, and/or parts, the elements, components, areas, layers, and/or parts cannot be limited by the terms. The terms can be used not to indicate specific sequences, top and bottom, or superiority and inferiority but to classify a certain element, area, or part from another element, area, or part. Thus, a first element, area, or part can be named a second element, area, or part without leaving from the right scope of the inventive concept.

The embodiments will now be described with reference to drawings in which ideal embodiments are schematically shown. In the drawings, modifications in shapes may be predicted according to, for example, manufacturing techniques and/or tolerances. Thus, the embodiments should not be construed as being limited to a specific shape in an area shown in the specification and should include modifications in shapes caused by manufacturing.

The present inventive concept will now be described in detail by describing exemplary embodiments with reference to the accompanying drawings. However, the present inventive concept may not be limited to the embodiments set forth herein, and may be embodied in many different forms; rather, these embodiments are provided so that this disclosure will be complete, and will fully convey the scope of the present inventive concept to those of ordinary skill in the art. In the drawings, the thicknesses of components may be exaggerated for convenience of description.

The terms used in the embodiments described below may have meaning commonly known in the technical field of the present inventive concept. For example, at least one indicates one at minimum, i.e., one or more number, and may be used as the same meaning with a singular or plural number.

Expressions such as “at least one of,” when preceding a list of elements, modify the entire list of elements and do not modify the individual elements of the list.

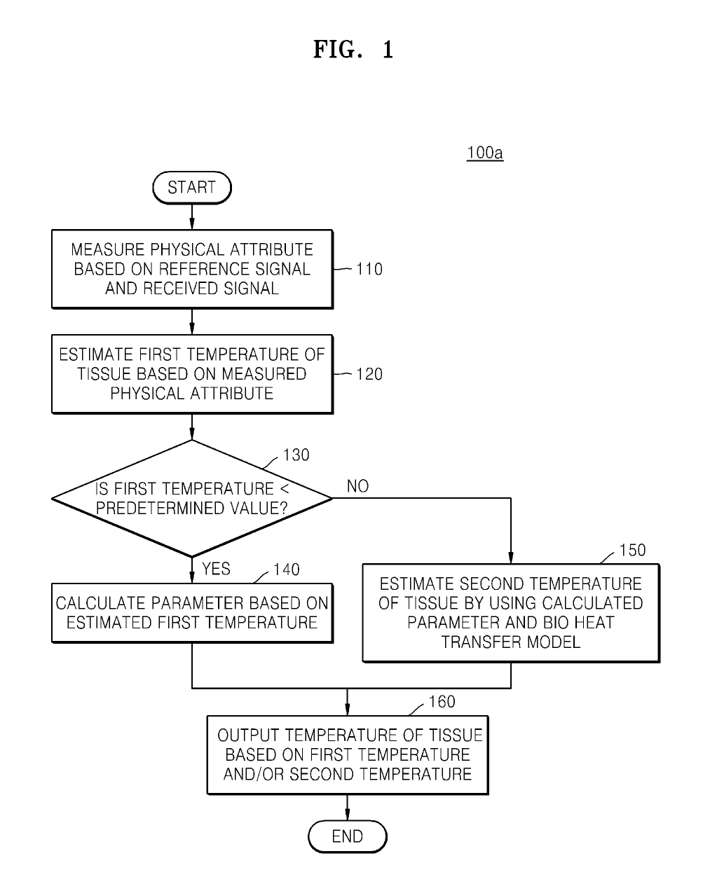

FIG. 1 is a flowchart illustrating a temperature estimation method 100a according to an embodiment.

In FIG. 1, the temperature estimation method 100a may include a measurement operation (110), a first estimation operation (120), a calculation operation (140), a second estimation operation (150), and an output operation (160).

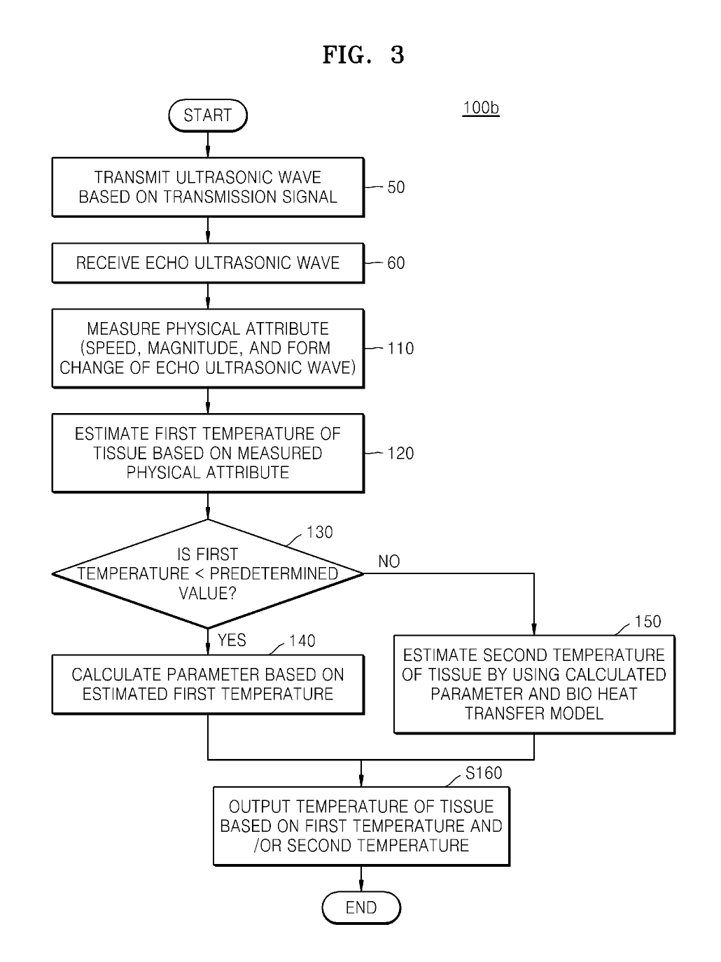

In the measurement operation (110), a physical attribute of a tissue may be measured based on a reference signal and a received signal. In more detail, during the measurement operation (110), the physical attribute may be measured based on a variation degree of the received signal compared with the reference signal. The physical attribute may include data regarding a temperature variation in the tissue. For example, to perform the measurement operation (110), at least one of a Harmonic Motion Imaging (HMI) method, a Change in Backscattered Energy (CBE) method, an echo shift method, and a nonlinear B/A parameter method may be used. An example of using the echo shift method to perform the measurement operation (110) will be described in more detail with reference to FIG. 3.

In the first estimation operation (120), a first temperature of the tissue may be estimated based on the physical attribute measured in the measurement operation (110). In more detail, during the first estimation operation (120), the first temperature of the tissue may be estimated based on the physical attribute (e.g., the data regarding the temperature variation in the tissue). For example, the physical attribute may be determined from a scattering coefficient according to temperature application, and the first temperature may be estimated using a variation of the scattering coefficient. After performing the first estimation operation (120), it may be determined in operation 130 whether the first temperature of the tissue is less than a predetermined value.

If the estimated first temperature is less than the predetermined value, it is determined that a temperature of the tissue is in a first range (e.g., a low-temperature range), and in this case, the first temperature estimated in the first estimation operation (120) that is performed in the first range may have high reliability. Thus, the output operation (160) of outputting a temperature of the tissue based on the first temperature that is an estimated value having high reliability may be performed.

If the estimated first temperature is less than the predetermined value, before performing the output operation (160), the calculation operation (140) of calculating a parameter based on the estimated first temperature may be performed. In the calculation operation (140), parameters to be used in a bio heat transfer model may be calculated based on the estimated first temperature. The bio heat transfer model represents a figure in which a temperature of a certain tissue varies, in a mathematical form, as shown in Equation 1.

$${ρ}_{t} {C}_{t} \frac{\partial T ( x , t )}{\partial t} = {k}_{t} {∇}^{2} T ( x , t ) + {V}_{{ρ}_{b}} {C}_{b} ( {T}_{b} - T ( x , t ) ) + Q ( x , t ) \qquad ( 1 )$$

In Equation 1, ρₜ denotes density of a tissue, Cₜ denotes specific heat of the tissue, T(x, t) denotes a temperature of the tissue,

$$\frac{\partial T ( x , t )}{\partial t} and {∇}^{2} T ( x , t )$$

denote temperature variations (i.e., a primary temporal differential value and a secondary spatial differential value) of the tissue according to time, kₜ denotes heat conductivity of the tissue, V_{ρ}_{_{b}} denotes density of a blood flow in the tissue, C_{b} denotes specific heat of the blood flow in the tissue, T_{b} denotes a temperature of the blood flow in the tissue, and Q(x, t) denotes heat added to the tissue from outside the tissue.

The first temperature of the tissue estimated in the first estimation operation (120) corresponds to T(x, t) as a temperature of the tissue. Further,

$$\frac{\partial T ( x , t )}{\partial t} and {∇}^{2} T ( x , t )$$

may be calculated based on the first temperature. By calculating the values (i.e., T(x, t)

$$\frac{\partial T ( x , t )}{\partial t} ,$$

and ∇²T(x,t)), parameters (e.g., ρₜ, Cₜ, kₜ, V_{ρ}_{_{b}}, C_{b}, T_{b}, and Q(x, t)) in the bio heat transfer model may be calculated. The calculated parameters may then be used in the second estimation operation (150).

Otherwise, if the estimated first temperature is equal to or greater than the predetermined value, the second estimation operation (150) of estimating a second temperature of the tissue by using the calculated parameter and the bio heat transfer model may be performed. If the estimated first temperature is equal to or greater than the predetermined value, it means that a temperature of the tissue is in a second range (e.g., a high-temperature range), and in this case, the first temperature estimated in the first estimation operation (120) may have low reliability. Thus, to more accurately estimate a temperature of the tissue, the second temperature of the tissue may be estimated using the second estimation operation (150) instead of the first estimation operation (120).

The parameter calculated in the calculation operation (140) may be used in the second estimation operation (150). In more detail, a temperature model of the tissue may be generated by substituting the parameter calculated in the calculation operation (140) into the bio heat transfer model. The bio heat transfer model may be modeled from a removed tissue in an experimental environment, i.e., an in vitro tissue, or a tissue in a live body, i.e., an in vivo tissue. By obtaining a solution of the temperature model (i.e., the bio heat transfer model) of the tissue, a temperature variation function of the tissue according to time may be obtained. The second temperature of the tissue may be estimated based on the temperature variation function.

If the estimated first temperature is equal to or greater than the predetermined value, the second temperature estimated in the second estimation operation (150) may be more accurate than the first temperature estimated in the first estimation operation (120). Thus, in the output operation (160) after the second estimation operation (150), a temperature of the tissue may be output based on the second temperature, which is an estimated value having high reliability.

Alternatively, in the output operation (160), a temperature of the tissue may be output based on both the first temperature and the second temperature. That is, if the estimated first temperature is equal to or greater than the predetermined value, in the output operation (160), an estimated value obtained by modifying the first temperature based on the second temperature estimated in the second estimation operation (150) may be calculated, and the calculated estimated value may be output as a temperature of the tissue.

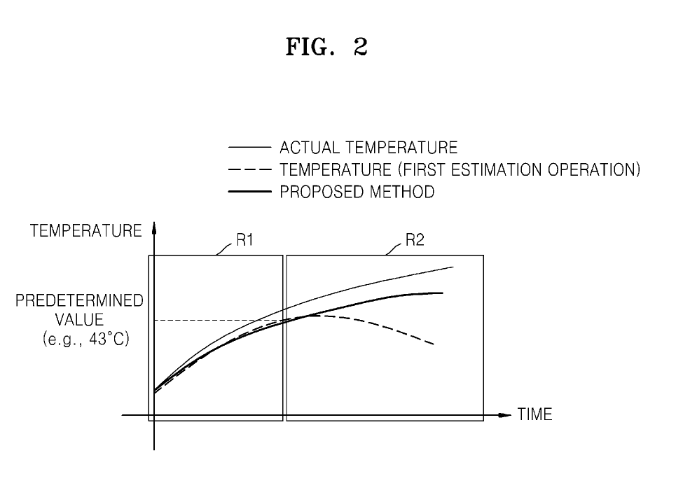

FIG. 2 is a graph showing temperatures of the tissue, which are estimated using the temperature estimation method 100a of FIG. 1.

In FIGS. 1 and 2, a physical attribute may be measured based on the reference signal and a received signal in the measurement operation (110), and the first temperature of the tissue may be estimated based on the physical attribute obtained in the first estimation operation (120).

For example, to detect temperatures of a lesion tissue and its surrounding tissues while performing a non-invasive therapy operation of removing the lesion tissue by heating a live tissue, such as tissue in a human body or another organism, for example, the temperature estimation method may be used. In this case, while performing the non-invasive therapy operation (e.g., heating an in vivo tissue), the measurement operation (110) and the first estimation operation (120) may be performed.

A first range R1 of FIG. 2 shows a figure in which the first temperature of the tissue estimated in the first estimation operation (120) is output. In more detail, the first range R1 shows a figure in which a temperature of the tissue is estimated using an echo shift method when the tissue is in a low-temperature state (such as lower than approximately 43° C., for example). This figure may correspond to a process of the initial stage of heating the tissue in the beginning of the non-invasive therapy operation.

It may be determined in the tissue in the low-temperature state (such as lower than approximately 43° C., for example) that a difference between an actual temperature of the tissue and the first temperature estimated in the first estimation operation (120) is not large. Thus, in the first range R1 (i.e., a low-temperature range), a temperature of the tissue may be output based on the first temperature, which is an estimated value having high reliability. In addition, the calculation operation (140) of calculating a parameter based on the first temperature may be performed.

A second range R2 of FIG. 2 shows a figure in which the second temperature of the tissue estimated in the second estimation operation (150) is output. In more detail, the second range R2 shows a figure in which a temperature of the tissue is estimated using the parameter calculated in the calculation operation (140) and the bio heat transfer model when the tissue is in a high-temperature state (e.g., equal to or higher than approximately 43° C., for example). This figure may correspond to a process in which the temperature of the tissue exceeds the predetermined value (such as approximately 43° C., for example) while continuing the non-invasive therapy operation.

It may be determined in the tissue in the high-temperature state (such as equal to or higher than approximately 43° C., for example) that a difference between an actual temperature of the tissue and the first temperature estimated in the first estimation operation (120) becomes larger as time elapses. Thus, in the second range R2 (i.e., a high-temperature range), a temperature of the tissue may be output based on the second temperature estimated in the second estimation operation (150) instead of the first temperature estimated in the first estimation operation (120).

FIG. 3 is a flowchart illustrating a temperature estimation method 100b according to an embodiment. The temperature estimation method 100b may be a modified example of the temperature estimation method 100a of FIG. 1. Hereinafter, repeated descriptions between the embodiments in FIGS. 1 and 3 are omitted.

In FIG. 3, an echo shift method may be used to perform the measurement operation (110) and the first estimation operation (120). The echo shift method may be defined by a method of estimating a temperature of a tissue based on a variation degree of a speed of an echo ultrasonic wave.

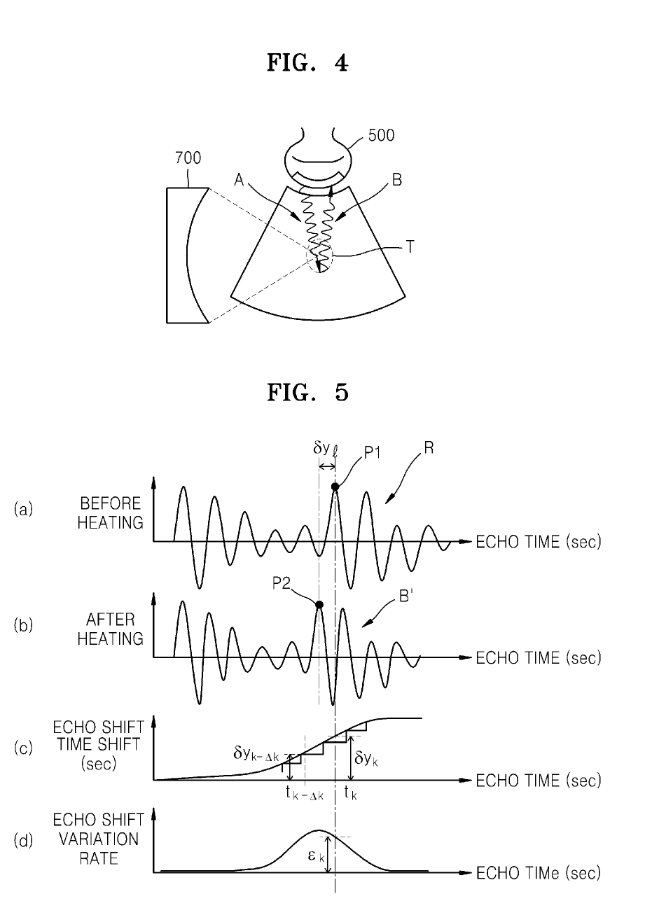

First, a transmission operation (50) of transmitting an ultrasonic wave A towards a tissue T may be performed, and then, a reception operation (60) of receiving an echo ultrasonic wave B may be performed. FIG. 4 is a schematic diagram of an ultrasonic treatment device and a temperature estimation apparatus according to embodiments. In FIG. 4, a figure of the transmission operation (50) in which the ultrasonic wave A is transmitted from a temperature estimation apparatus 500 and radiated to the tissue T and a figure of the reception operation (60) in which the echo ultrasonic wave B generated by reflecting the ultrasonic wave A from the tissue T is received by the temperature estimation apparatus 500 are shown. The received echo ultrasonic wave B may be converted to a received signal B′.

Thereafter, the measurement operation (110) of measuring a physical attribute based on a reference signal R and the received signal B′ is performed. For example, the physical attribute may be determined from a speed variation (hereinafter, refer to as ‘echo shift’) of the received signal B′ converted from the echo ultrasonic wave B compared with the reference signal R. The reference signal R may be a signal converted from an echo ultrasonic wave reflected from the tissue T before a temperature of the tissue T is changed.

If the tissue T is heated by an ultrasonic treatment device 700, a temperature of the tissue T increases, and accordingly, an ultrasonic wave transfer speed in the tissue T varies. The variations of the ultrasonic wave transfer speed in the tissue T are shown in FIGS. 5A and 5B. FIG. 5A is a graph showing an echo ultrasonic wave received before a temperature of a tissue increases, and FIG. 5B is a graph showing an echo ultrasonic wave received after the temperature of the tissue increases. As shown in FIGS. 5A and 5B, a position of a first peak P1 of the reference signal R before heating the tissue T is different from a position of a second peak P2 of the received signal B′ after heating the tissue T.

This position difference is caused because a transfer speed of the echo ultrasonic wave B reflected from the tissue T varies as a temperature of the tissue T varies. Thus, the echo shift may be measured based on the received signal B′ and the reference signal R. FIG. 5C is a graph showing a result obtained by measuring the echo shift, and FIG. 5D is a graph showing a variation rate of the echo shift.

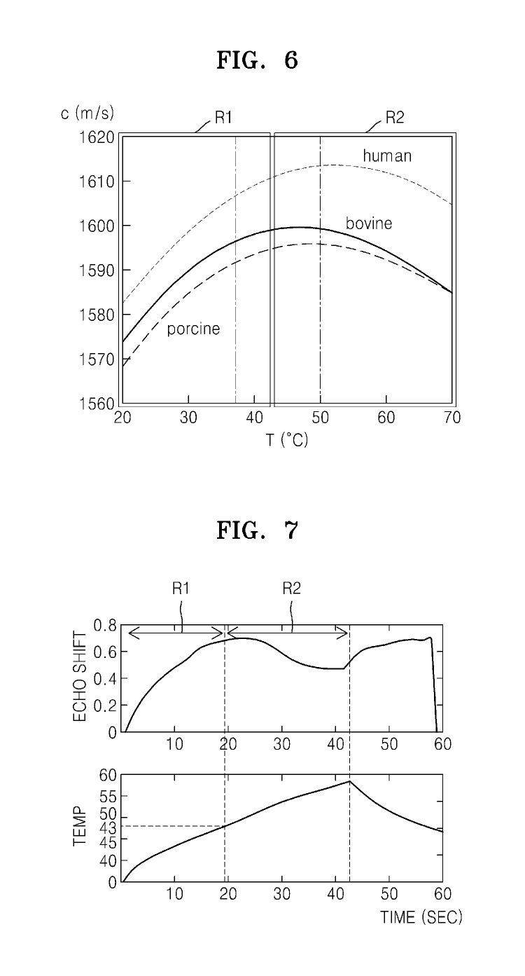

In FIG. 3, the physical attribute, such as the echo shift, for example, is measured in the measurement operation (110), and the first estimation operation (120) of estimating the first temperature of the tissue T based on the physical attribute is performed. FIG. 6 is a graph showing a relationship between temperatures of tissues and velocities of propagation of echo ultrasonic waves. In FIG. 6, in the first range R1 (e.g., a low-temperature range), a speed variation of an echo ultrasonic wave is proportional to a variation degree of a temperature of the tissue T.

By using the proportional relationship, the first temperature, which is an estimated value of a temperature of the tissue T, may be calculated. FIG. 7 is a graph showing the first temperature estimated based on the echo shift. In the first range R1 (e.g., a low-temperature range), i.e., in a time interval from approximately 0 second to approximately 20 seconds, the echo shift increases, and accordingly, the first temperature may be calculated.

In a second range R2 (e.g., a high-temperature range) of FIG. 6, a speed variation of the echo ultrasonic wave may not be proportional to a variation degree of a temperature of the tissue T, unlike the first range R1. Thus, the first temperature calculated in the second range R2 (e.g., a high-temperature range), i.e., in a time interval from approximately 20 seconds to approximately 30 seconds, may not be used to estimate a temperature of the tissue T.

In FIG. 3, after estimating the first temperature in the first estimation operation (120), if the first temperature is less than the predetermined value, the calculation operation (140) of calculating a parameter of the bio heat transfer model by using the first temperature may be performed. For example, the parameter may include at least one of a heat conductivity of the tissue T and heat applied to the tissue T.

To simplify an expression, when the possibility of heat diffusion due to a blood flow in the tissue T is excluded, Equation 1 may be expressed by Equation 2.

$${ρ}_{t} {C}_{t} \frac{\partial T ( x , t )}{\partial t} = {k}_{t} {∇}^{2} T ( x , t ) + Q ( x , t ) \qquad ( 2 )$$

Equation 3 may be obtained by rearranging Equation 2.

$$\frac{\partial T ( x , t )}{\partial t} = {k}_{t}^{′} {∇}^{2} T ( x , t ) + {Q}^{′} ( x , t ) \\ where , {k}_{t}^{′} = \frac{{k}_{t}}{{ρ}_{t} {C}_{t}} , {Q ( x , t )}^{′} = \frac{Q ( x , t )}{{ρ}_{t} {C}_{t}} \qquad ( 3 )$$

During the calculation operation (140), a heat conductivity kₜ′ of the tissue T and heat Q(x, t)′ applied to the tissue T may be calculated.

To calculate these parameters, a temperature T(x, t), temperature variations

$$\frac{\partial T ( x , t )}{\partial t} ,$$

and ∇²T(x,t) may first be calculated based on the first temperature estimated in the first range R1 (e.g., a low-temperature range). The calculated values may be substituted into a bio heat transfer model based on Equation 3, and as a result, intermediate expressions with respect to the heat conductivity kₜ′ of the tissue T and heat Q(x, t)′ applied to the tissue T may be generated.

By obtaining a solution of the intermediate expressions, parameters, such as the heat conductivity kₜ′ of the tissue T and heat Q(x, t)′ applied to the tissue T, may be calculated.

The parameter calculation based on Equations 2 and 3 is performed by excluding the possibility of heat diffusion due to a blood flow in the tissue T. However, if the possibility of heat diffusion is considered, Equation 1 may be expressed by Equation 4.

$$\frac{\partial T ( x , t )}{\partial t} = {k}_{t}^{′} {∇}^{2} T ( x , t ) + {( {V}_{{ρ}_{b}} {C}_{b} )}^{′} ( {T}_{b} - T ( x , t ) ) + {Q}^{′} ( x , t ) \\ where , {k}_{t}^{′} = \frac{{k}_{t}}{{ρ}_{t} {C}_{t}} , {Q ( x , t )}^{′} = \frac{Q ( x , t )}{{ρ}_{t} {C}_{t}} , ( {V}_{{ρ}_{b}} {C}_{b} ) , = \frac{{V}_{{ρ}_{b}} {C}_{b}}{{ρ}_{t} {C}_{t}} \qquad ( 4 )$$

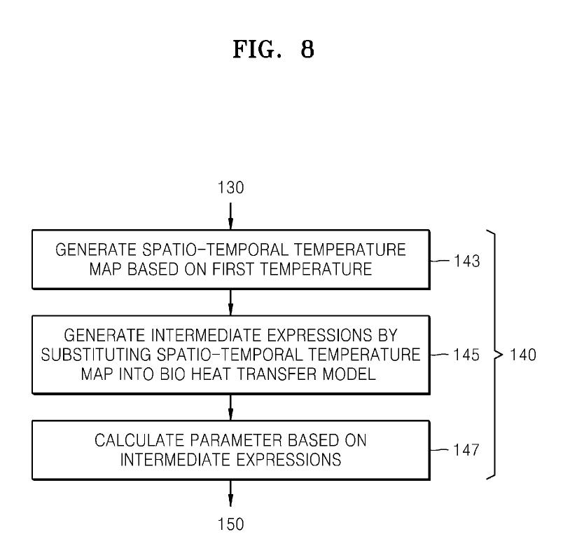

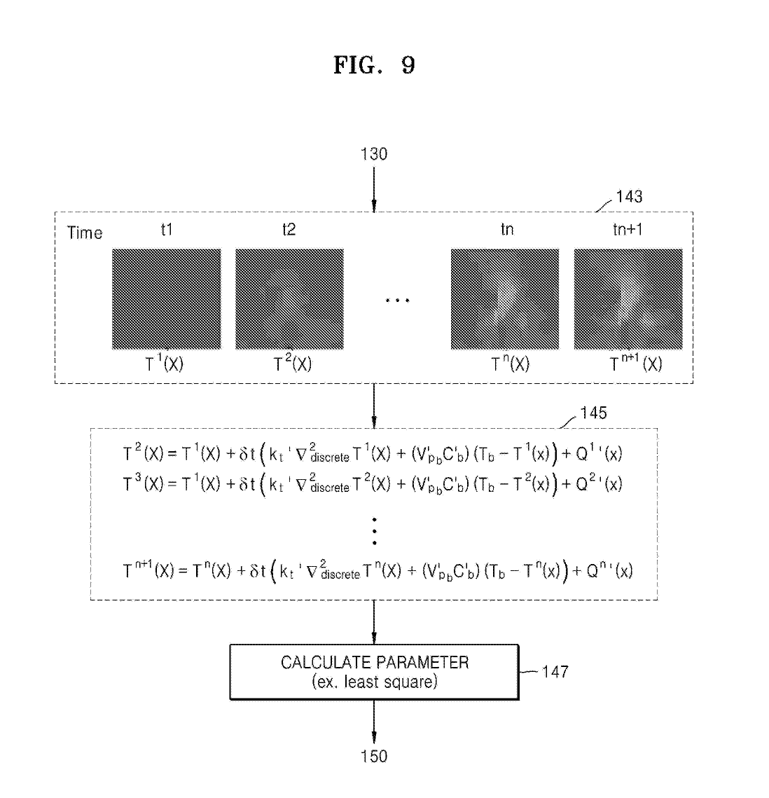

FIGS. 8 and 9 show the calculation operation (140 of FIG. 3) in more detail based on Equation 4. In FIG. 8, in operation 143, a plurality of spatio-temporal temperature maps may be generated based on the first temperature estimated in the first estimation operation (120 of FIG. 3). For example, as shown in FIG. 9, a first spatio-temporal temperature map T¹(X) may be generated based on a first temperature estimated at a first time t1, and a second spatio-temporal temperature map T²(X) may be generated based on a first temperature estimated at a second time t2.

In operation 145, intermediate expressions may be generated by substituting spatio-temporal temperature maps into the bio heat transfer model. For example, a first intermediate expression, such as Equation 5, may be generated by substituting the second spatio-temporal temperature map T²(X) and the first spatio-temporal temperature map T¹(X) into the bio heat transfer model.

T²(x)=T¹(x)+δt(kₜ′∇²T¹(x)+(V′_{ρ}_{_{b}}C′_{b})(T_{b}−T¹(x))+Q¹′(x)) (5)

Likewise, an nth intermediate expression, such as Equation 6, may be generated by substituting an (n+1)th spatio-temporal temperature map Tⁿ⁺¹(X) and an nth spatio-temporal temperature map Tⁿ(X) into the bio heat transfer model.

Tⁿ⁺¹(x)=Tⁿ(x)+δt(kₜ′∇²Tⁿ(x)+(V′_{ρ}_{_{b}}C′_{b})(T_{b}−Tⁿ(x))+Qⁿ′(x)) (6)

In operation 147, parameters may be calculated based on the intermediate expressions, such as Equations 5 and 6. For example, the parameters may be calculated by applying a least square method to the intermediate expressions. However, the present inventive concept is not limited thereto, and the parameters may also be calculated using methods other than the least square method.

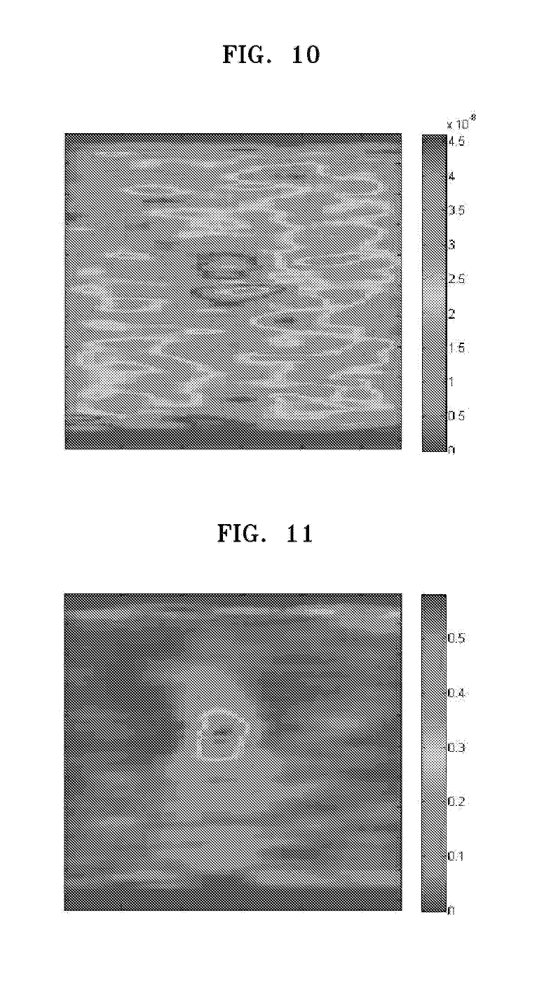

The parameters calculated based on the intermediate expressions are illustrated in FIGS. 10 and 11. FIG. 10 illustrates a parameter of the heat conductivity kₜ′ of the tissue T, and FIG. 11 illustrates the heat Q(x, t)′ applied to the tissue T.

In FIG. 3, after performing the calculation operation (140), the output operation (160) is performed. If the first temperature estimated in the first estimation operation (120) is less than the predetermined value, the first temperature having high reliability may be output as a temperature of the tissue T in the output operation (160).

If the first temperature estimated in the first estimation operation (120) is equal to or greater than the predetermined value, the second estimation operation (150) may be performed. In the second estimation operation (150), a parameter calculated based on the first temperature estimated in the first range R1 (e.g., a low-temperature range) and the bio heat transfer model may be used. For example, the second temperature may be estimated using the parameters shown in FIGS. 10 and 11 and the bio heat transfer model by Equation 6. After performing the second estimation operation (150), the output operation (160) is performed. In the output operation (160), the second temperature having high reliability may be output as a temperature of the tissue T.

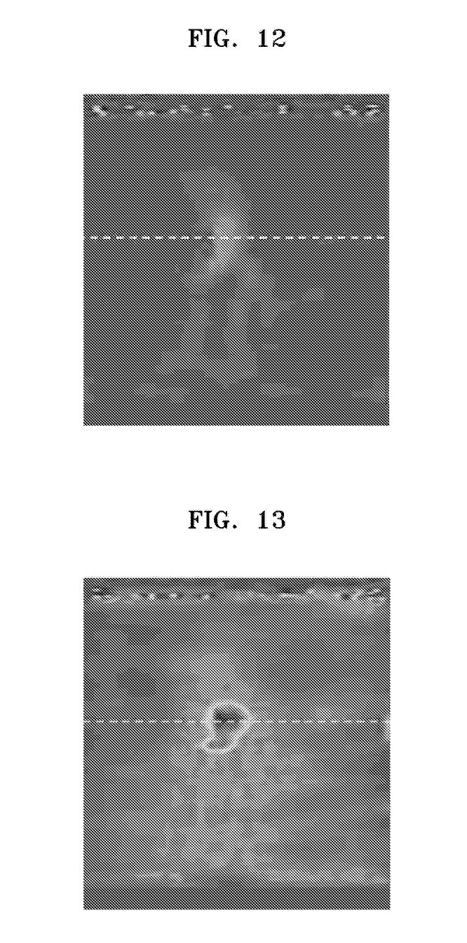

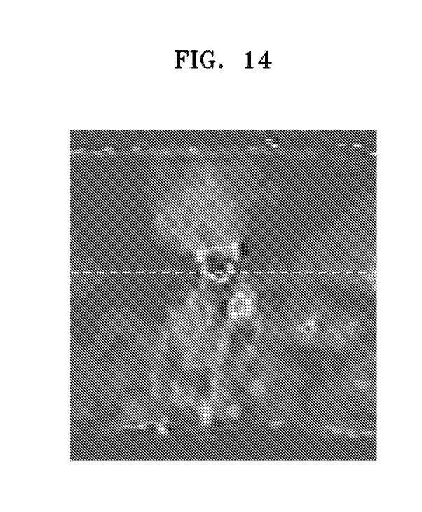

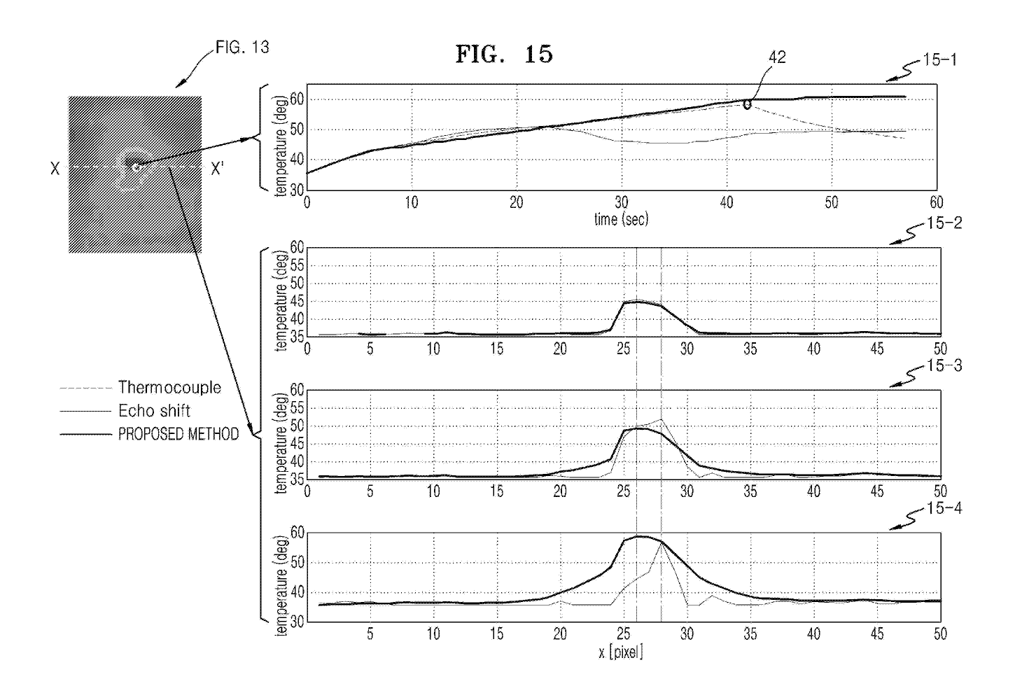

FIGS. 12 and 13 illustrate results obtained by estimating a temperature of the tissue T by using the temperature estimation method 100b according to an embodiment.

FIG. 12 shows an image in which a temperature of the tissue T is output by using the first estimation operation (120) when the temperature of the tissue T is in the first range R1 (e.g., a low-temperature range), and FIG. 13 shows an image in which a temperature of the tissue T is output by using the second estimation operation (150) when the temperature of the tissue T is in the second range R2 (e.g., a high-temperature range).

In FIG. 12, the first estimation operation (120 of FIG. 3) of estimating the first temperature of the tissue T by using an estimation method, such as the echo shift method, for example, is performed, and if the estimated first temperature is less than the predetermined value, the output operation (160 of FIG. 3) of outputting the first temperature as a temperature of the tissue T is performed. The first estimation operation (120 of FIG. 3) and the output operation (160 of FIG. 3) may be performed at the same time as the non-invasive therapy operation of heating the tissue T, and accordingly, a temperature variation of the tissue T during the non-invasive therapy operation may be detected in real-time.

In FIG. 13, if the first temperature estimated in the first estimation operation (120 of FIG. 3) is equal to or greater than the predetermined value, the second estimation operation (150 of FIG. 3) of estimating the second temperature by using the parameter calculated in the calculation operation (140 of FIG. 3) and the bio heat transfer model is performed, and the output operation (160 of FIG. 3) of outputting the second temperature as a temperature of the tissue T is performed. The second estimation operation (150 of FIG. 3) and the output operation (160 of FIG. 3) may be performed at the same time as the non-invasive therapy operation of heating the tissue T, and accordingly, a temperature variation of the tissue T during the non-invasive therapy operation may be detected in real-time.

In the first estimation operation (120 of FIG. 3), such as the echo shift method, for example, when a temperature of the tissue T is low, more correct temperature estimation is possible than other methods. However, when a temperature of the tissue T is high, accuracy of temperature estimation may be low. FIG. 14 illustrates a figure in which a temperature estimated by using the first estimation operation (120 of FIG. 3) is output when a temperature of the tissue T is high. Since a result of FIG. 14 does not clearly show temperatures of a lesion tissue and its surrounding tissues, accuracy of the result of FIG. 14 is lower than that of a result of FIG. 13.

The temperature estimation method 100a or 100b according to an embodiment uses the first estimation operation (e.g. the echo shift method) having high reliability in the first range (e.g., a low-temperature range) and uses, in the second range (e.g., a high-temperature range), the second estimation operation of estimating a temperature of a tissue by modeling a bio heat transfer model based on a result measured in a low temperature. Thus, relatively correct temperature estimation result may be obtained by outputting a first temperature using the first estimation operation having high reliability in the first range and outputting a second temperature using the second estimation operation performed using the bio heat transfer model modeled based on the first temperature in the second range.

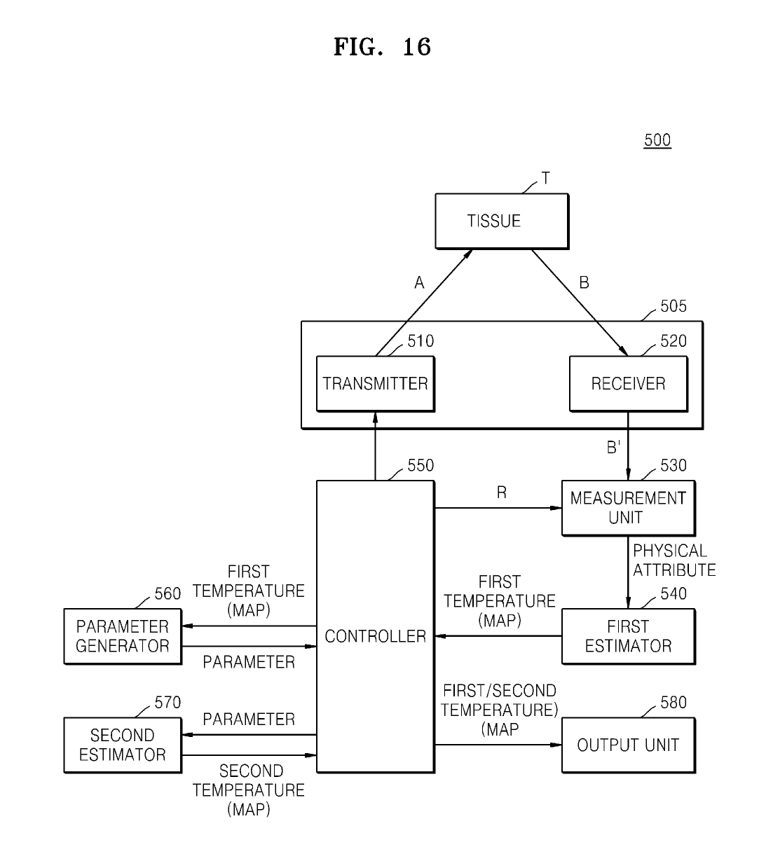

FIG. 15 illustrates graphs showing the temperature estimation result of FIG. 13 in more detail. A first graph 15-1 shows a figure in which a temperature of the tissue T varies according to time. A second graph 15-2 shows a temperature distribution of the tissue T at t=10 s, i.e., when a temperature of the tissue T is approximately 45° C. A third graph 15-3 shows a temperature distribution of the tissue T at t=20 s, i.e., when a temperature of the tissue T is approximately 50° C. A fourth graph 15-4 shows a temperature distribution of the tissue T at t=42 s, i.e., when a temperature of the tissue T is approximately 60° C. A temperature map 13 shows a temperature distribution of the tissue T at t=42 s in the first graph 15-1, and the second graph 15-2 corresponds to a temperature distribution along X-X′ of the temperature map 13.

In the first graph 15-1 and the second graph 15-2, when a temperature of the tissue T is less than 45° C., a slight difference exists between a temperature estimated in the first estimation operation (e.g., the echo shift method) and an actually measured temperature. That is, in the first range (e.g., a low-temperature range), the first estimation operation may provide a temperature estimation result having high reliability.

In the first graph 15-1 and the third graph 15-3, when a temperature of the tissue T is approximately 50° C., a small difference exists between the first temperature estimated in the first estimation operation (e.g., the echo shift method) and an actually measured temperature. Thus, the second temperature estimated in the second estimation operation (e.g., a temperature modeling method) may be output as a temperature estimation result of the tissue T.

In the first graph 15-1 and the fourth graph 15-4, when a temperature of the tissue T is approximately 60° C., a large difference exists between the first temperature estimated in the first estimation operation (e.g., the echo shift method) and an actually measured temperature. Thus, the second temperature estimated in the second estimation operation (e.g., the temperature modeling method) may be output as a temperature estimation result of the tissue T.

FIG. 16 is a block diagram of a temperature estimation apparatus 500 according to an embodiment. For example, the temperature estimation apparatus 500 may be configured to estimate a temperature of the tissue T by using the temperature estimation method 100a or 100b of FIG. 1 or FIG. 3. Hereinafter, repeated descriptions will be omitted.

In FIG. 16, the temperature estimation apparatus 500 may include a transmitter 510, a receiver 520, a measurement unit 530, a first estimator 540, a controller 550, a parameter generator 560, a second estimator 570, and an output unit 580.

The transmitter 510 may be configured to radiate an ultrasonic wave A towards the tissue T to estimate a temperature of the tissue T. The receiver 520 may be configured to receive an echo ultrasonic wave B generated by reflecting the ultrasonic wave A radiated towards the tissue T. For example, the transmitter 510 and the receiver 520 may be implemented by a transducer 505.

The measurement unit 530 may be configured to receive a received signal B′ from the receiver 520, receive a reference signal R from the controller 550, and measure a physical attribute based on the received signal B′ and the reference signal R. When the echo shift method is used, the physical attribute may be determined from a speed variation, i.e., an echo shift, of the received signal B′ compared with the reference signal R, as described above.

The first estimator 540 may be configured to receive information regarding the physical attribute from the measurement unit 530 and estimate a first temperature of the tissue T. The estimated first temperature may be provided to the controller 550.

The controller 550 may be configured to control the temperature estimation apparatus 500. For example, the controller 550 may be configured to provide a transmission signal to the transmitter 510 so that the transmitter 510 generates the ultrasonic wave A and to provide the reference signal R to the measurement unit 530. In addition, the controller 550 may receive the first temperature from the first estimator 540, determine whether the first temperature is less than the predetermined value, and control the parameter generator 560, the output unit 580, and/or the second estimator 570 according to a result of the determination.

If the first temperature provided by the first estimator 540 is less than the predetermined value, the controller 550 may output temperature information of the tissue T to the output unit 580 based on the first temperature, and the output unit 580 may output a temperature of the tissue T based on the temperature information. In addition, the controller 550 may provide the first temperature that is information for generating a parameter to the parameter generator 560. The parameter generator 560 may calculate a parameter used in a bio heat transfer model based on the first temperature and provide the parameter to the controller 550.

If the first temperature provided by the first estimator 540 is equal to or greater than the predetermined value, the controller 550 may provide the parameter to the second estimator 570. The second estimator 570 may estimate a second temperature of the tissue T according to time by substituting the parameter into the bio heat transfer model and provide the estimated second temperature to the controller 550. Thereafter, the controller 550 may provide temperature information of the tissue T to the output unit 580 based on the second temperature, and the output unit 580 may output a temperature of the tissue T based on the temperature information.

It should be understood that a shape of each part in the drawings attached to clearly understand the present inventive concept is illustrative. It should be noted that the shown shape may be modified to various shapes. Like reference numerals in the drawings denote like elements.

As described above, according to the one or more of the above embodiments, a temperature estimation method and a temperature estimation apparatus may accurately estimate a necrosis level of a tissue and the safety of normal tissues by obtaining a temperature distribution of a treatment part and its surroundings, resulting in safe and efficient necrosis of a tumor.

The above-described embodiments may be recorded in computer-readable media including program instructions to implement various operations embodied by a computer. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The computer-readable media may also be a distributed network, so that the program instructions are stored and executed in a distributed fashion. The program instructions may be executed by one or more processors. The computer-readable media may also be embodied in at least one application specific integrated circuit (ASIC) or Field Programmable Gate Array (FPGA), which executes (processes like a processor) program instructions. Examples of program instructions include both machine code, such as produced by a compiler, and files containing higher level code that may be executed by the computer using an interpreter. The above-described devices may be configured to act as one or more software modules in order to perform the operations of the above-described embodiments, or vice versa.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A temperature estimation method comprising:
measuring a physical attribute of a tissue;
a first estimation operation to estimate a first temperature of the tissue based on the physical attribute;
calculating, by a processor, at least one parameter used in a bio heat transfer model based on the estimated first temperature; and
a second estimation operation to estimate a second temperature of the tissue by using the calculated at least one parameter and the bio heat transfer model.

2. The temperature estimation method of claim 1, wherein the first estimation operation is performed when a temperature of the tissue exists in a first range, and
the second estimation operation is performed when the temperature of the tissue exists in a second range greater than the first range.

3. The temperature estimation method of claim 1, wherein the second estimation operation is performed when the first temperature estimated in the first estimation operation is greater than a predetermined value.

4. The temperature estimation method of claim 1, further comprising removing a lesion by heating the tissue,
wherein at least one of the first estimation operation and the second estimation operation is performed at the same time as the removing of the lesion.

5. The temperature estimation method of claim 1, wherein the at least one parameter includes at least one of heat conductivity of the tissue and heat applied to the tissue from the outside of the tissue.

6. The temperature estimation method of claim 1, wherein, in the parameter calculation, the at least one parameter is calculated by substituting the estimated first temperature that is less than a predetermined value and a variation of the temperature into the bio heat transfer model.

7. The temperature estimation method of claim 1, wherein the parameter calculation comprises:
generating a spatio-temporal temperature map of the tissue based on the first temperature of the tissue; and
calculating the at least one parameter based on intermediate expressions obtained by substituting the spatio-temporal temperature map into the bio heat transfer model.

8. The temperature estimation method of claim 7, wherein the at least one parameter is calculated by applying a least square method to the intermediate expressions.

9. The temperature estimation method of claim 1, wherein the physical attribute measurement is performed using at least one of a method of estimating a temperature by using a temperature sensor and a method of measuring a speed variation, a magnitude variation, and a form change due to a nonlinear characteristic with respect to an ultrasound signal.

10. The temperature estimation method of claim 1, wherein the physical attribute measurement comprises:
radiating an ultrasonic wave to a predetermined area corresponding to the tissue of a person to be diagnosed;
receiving an echo ultrasonic wave reflected by the tissue; and
converting the echo ultrasonic wave to the received signal.

11. The temperature estimation method of claim 10, wherein the physical attribute measurement is performed using an echo shift method, and
the physical attribute is determined by a speed variation between the reference signal and the received signal.

12. The temperature estimation method of claim 1, wherein the second estimation operation comprises:
generating a temperature model of the tissue by substituting the calculated at least one parameter into the bio heat transfer model; and
estimating the second temperature of the tissue based on the temperature model.

13. A temperature estimation method comprising:
radiating an ultrasonic wave to a predetermined area corresponding to a tissue of a person to be diagnosed;
receiving an echo ultrasonic wave reflected by the tissue;
converting the echo ultrasonic wave to a received signal;
measuring a variation between a reference signal and the received signal;
a first estimation operation of estimating a first temperature of the tissue based on the variation;
calculating, by a processor, at least one parameter used in a bio heat transfer model based on the estimated first temperature; and
a second estimation operation of estimating a second temperature of the tissue by using the at least one parameter and the bio heat transfer model, the second estimation operation being performed when the estimated first temperature is greater than a predetermined value.

14. The temperature estimation method of claim 13, further comprising outputting a temperature of the tissue based on at least one of the first temperature and the second temperature.

15. The temperature estimation method of claim 1, wherein, in the temperature output, the first temperature is output as the temperature of the tissue when the estimated first temperature is less than the predetermined value, and the second temperature is output as the temperature of the tissue when the estimated first temperature is equal to or greater than the predetermined value.

16. A temperature estimation apparatus comprising:
a measurement unit to measure a physical attribute of a tissue based on a reference signal and a received signal;
a first estimation unit to estimate a first temperature of the tissue based on the physical attribute; and
a second estimation unit to estimate a second temperature of the tissue by using a bio heat transfer model modeled based on the estimated first temperature.

17. The temperature estimation apparatus of claim 16, further comprising:
a parameter generator to calculate a parameter based on the estimated first temperature.

18. The temperature estimation apparatus of claim 17, further comprising:
an output unit to output a temperature of the tissue based on at least one of the first temperature and the second temperature.

19. The temperature estimation apparatus of claim 16, further comprising:
a transmitter to radiate an ultrasonic wave to a predetermined area corresponding to a tissue of a person to be diagnosed;
a receiver to receive an echo ultrasonic wave reflected by the tissue; and
a converter to convert the echo ultrasonic wave to a received signal.

20. A non-transitory computer-readable recording medium storing a program to implement the method of claim 1.

21. A temperature estimation method comprising:
measuring a physical attribute of a tissue;
estimating, by a processor, a first temperature of the tissue based on the measured physical attribute;
determining if the first temperature is below a predetermined value; and
selectively outputting the first temperature if the first temperature is below the predetermined value, or if the first temperature is not below the predetermined value, outputting a second temperature calculated from a bio heat transfer model.

22. The temperature estimation method of claim 21, wherein the bio heat transfer model uses the first temperature as a parameter.
